(19) United States
(12) Patent Application Publication
(10) Pub. No.: US 2017/0128014 A1
(43) Pub. Date: May 11, 2017

(54) IMPLANTABLE DEVICES AND METHODS FOR MONITORING COPD IN PATIENTS

(71) Applicant: THE ALFRED E. MANN FOUNDATION FOR SCIENTIFIC RESEARCH, Valencia, CA (US)

(72) Inventors: Morten Hansen, Welwyn (GB); Desmond B. Keenan, Valencia, CA (US)

(73) Assignee: THE ALFRED E. MANN FOUNDATION FOR SCIENTIFIC RESEARCH, Valencia, CA (US)

(21) Appl. No.: 15/343,763

(22) Filed: Nov. 4, 2016

Related U.S. Application Data
(60) Provisional application No. 62/251,395, filed on Nov. 5, 2015.

(51) Int. Cl. A61B 5/00; A61B 5/0205; A61B 5/04; A61B 5/07; A61B 5/0488

(52) U.S. Cl. CPC A61B 5/6867; A61B 5/076; A61B 5/0488; A61B 5/04018; A61B 5/7203; A61B 5/0205; A61B 5/746; A61B 5/02405

(57) ABSTRACT

A medical monitoring system and method for monitoring a patient is provided. A sensor is implanted within the patient. A biomarker is detected via the implanted sensor within the patient. The detected biomarker is indicative of a neural respiratory drive (NRD) of the patient. An NRD index value is generated based on the detected biomarker.

### RELATED APPLICATION

This application claims priority from U.S. Provisional Patent Application Ser. No. 62/251,395, filed Nov. 5, 2015, which is expressly incorporated herein by reference.

### FIELD OF THE INVENTION

The present invention relates to medical systems, and in particular, medical systems for monitoring a respiratory disease, such as Chronic Obstructive Pulmonary Disease (COPD), in patients.

### BACKGROUND OF THE INVENTION

Chronic Obstructive Pulmonary Disease (COPD) is a type of obstructive lung disease characterized by long-term poor airflow, resulting from the narrowing of small airways and breakdown of lung tissue. COPD is the fourth leading cause of death in the United States, with approximately twenty percent of adult Americans suffering from this disease. COPD develops as a significant and chronic inflammatory response to inhaled irritants, with the most common cause being tobacco smoking. The main symptoms of COPD include shortness of breath, productive cough, and sputum production, making common activities, such as walking upstairs and carrying objects, difficult to perform. There is no known cure for COPD, with the symptoms present for a prolonged period of time and worsening over time.

Outpatient management of patients with stable COPD involves improving quality of life by relieving symptoms, slowing the progressive deterioration of lung function improving quality of life by preventing acute exacerbations (a sudden worsening of symptoms). Acute exacerbation of COPD may present with signs of increased work of breathing such as fast breathing, fast heart rate, seating, active use of muscles in the neck, a bluish tinge to the skin, and confusion or combative behavior in very severe exacerbations. An acute exacerbation is commonly triggered by infection or environmental pollutants, or sometimes by other factors such as improper use of medications. Those with many exacerbations have a faster rate of deterioration of their lung function. Oftentimes, patients having actuate exacerbations of COPD require hospitalization.

It is known to monitor a COPD patient for the purpose of predicting treatment failure, clinical deterioration, and re-admission to the hospital. For example, it is known to measure parasternal electromyogram (EMG) signals during normal respiratory cycles of the patient using a medical monitoring system that utilizes electrodes that are attached to the chest of the patient. Such EMG signals are indicative of a neural respiratory drive (NRD) of the patient, which is the output of the central nervous system's respiratory center and which correlates to the respiratory effort of the patient. Because it is known that the NRD measurement acquired from the ribcage region generally increases as the COPD symptoms increase, as a result of increased chest effort in response to chest tightening. Because EMG signals indicative of the extent of the COPD may vary from patient-to-patient, a calibration process is typically performed by normalizing the measured EMG signals to the patient. This can be accomplished by, e.g., comparing each measured EMG signal to a patient-specific reference (e.g., an EMG signal measured during maximum respiratory effort of that patient).

Although this medical monitoring system provides a suitable means for monitoring COPD patients, it has some downfalls. For example, in such a medical monitoring system, the electrodes must be manually attached to the patient's chest. As such, at least for outpatient monitoring, the patient must apply the electrodes, thereby imposing additional tasks on the patient and requiring patient compliance for the medical monitoring system to be effective. Furthermore, medical monitoring systems that utilize externally attached electrodes suffer from signal reliability and robustness issues due to electrode liftoff, skin impedance changes over the course of the day, movement artifacts, lack of repeatable electrode placement, as well as potential wire breakages. As a result, the patient-specific reference generated by such electrodes to which each EMG signal is compared must be periodically measured at these unstable electrodes to properly normalize each EMG signal. In this case where the patient-specific reference is an EMG signal during maximum respiratory effort, the patient is required to perform a series of maximum respiratory calibration sessions during the COPD monitoring process, thereby making patient compliance even more difficult to achieve.

There, thus, remains a need for providing a COPD monitoring system that does not require multiple calibrations and requires minimal to no effort for the patient.

### SUMMARY OF THE INVENTION

In accordance with a first aspect of the present inventions, a medical monitoring system for a patient is provided. The medical monitoring system comprises an implantable sensor device configured for detecting a biomarker over at least one respiratory cycle (e.g., during an inspiration of at least one resting respiratory cycle) of the patient, wherein the detected biomarker is indicative of a neural respiratory drive (NRD) of the patient. The sensor may be sized to be implanted adjacent an intercostal muscle of the patient. The medical monitoring system further comprises processing circuitry configured for generating an NRD index value based on the detected biomarker.

The detected biomarker may be, e.g., an electromyogram (EMG) signal. In this case, the processing circuitry may be further configured for processing the EMG signal. Processing the EMG signal may, e.g., comprise one of sampling the EMG signal, acquiring an envelope of the EMG signal, and integrating the EMG signal. Processing the EMG signal may, e.g., comprise filtering cardiac artifacts from the EMG signal and/or deriving one or more EMG data values from the EMG signal for each respiratory cycle by obtaining a peak value, and computing a root mean square (RMS) value, a quadratic mean value, or running average value of the EMG signal. If the biomarker is detected over a plurality of respiratory cycles, generating the NRD index value may further comprise obtaining the highest amplitude, median, or average of the EMG data values over the plurality of respiratory cycles.

In one embodiment, the processing circuitry is further configured for determining a heart rate variability and/or respiration rate variability of the patient from the detected biomarker, and determining a validity of the detected biomarker based on the determined heart rate variability and/or respiration rate variability. In another embodiment, the medical monitoring system further comprises memory, in which case, the processing circuitry configured for storing the NRD index value in the memory if the EMG signal is determined to be valid. In still another embodiment, the processing circuitry is configured for normalizing the NRD index value. For example, the processing circuitry may be further configured for obtaining a patient-specific reference value, wherein normalizing the NRD index value comprising computing a function of the NRD index value and the patient-specific reference value.

In yet another embodiment, the medical monitoring system further comprises a display configured for graphically displaying the NRD index value to the patient and/or generating an alert or notification signal in response to determining that the onset of the acute exacerbation of the respiratory disease in imminent. If the patient suffers from a respiratory disease (e.g., Chronic Obstructive Pulmonary Disease (COPD), the processing circuitry may be further configured for determining an extent of the respiratory disease based on the generated NRD index. In this case, the processing circuitry may be further configured for predicting an onset of an acute exacerbation of the respiratory disease in the patient based on the generated NRD index value.

For example, the processing circuitry may be configured for predicting the onset of the acute exacerbation of the respiratory disease in the patient comprises comparing the resting NRD index value to an absolute NRD threshold value, and determining that the onset of the acute exacerbation of the respiratory disease is imminent if the resting NRD index value exceeds the absolute NRD threshold. As another example, the processing circuitry may be configured for the onset of the acute exacerbation of the respiratory disease in the patient comprises computing an NRD index difference value between the resting NRD index value and a previously generated NRD index value, comparing the NRD difference value to a differential NRD threshold value, and determining that the onset of the acute exacerbation of the respiratory disease is imminent if the NRD index difference value exceeds the differential NRD threshold value.

The processing circuitry may be contained in one or more devices. For example, the medical monitoring system may further comprise an external control device containing at least a portion of the processing circuitry. As another example, the medical monitoring system may further comprise a personal wireless device configured for communicating with the external control device, in which case, the personal wireless device may contain another portion of the processing circuitry. As still another example, the medical monitoring system may further comprise a remote computer configured for communicating with the personal wireless device via a cloud network, in which case, the remote computer may contain still another portion of the processing circuitry.

In accordance with a second aspect of the present inventions, a method of monitoring a patient is provided. The method comprises implanting a sensor within the patient, e.g., in an intercostal muscle of the patient, such as the second parasternal intercostal muscle, or even the diaphragm of the patient. The method further comprises detecting a biomarker via the implanted sensor over at least one respiratory cycle (e.g., during an inspiration of at least one resting respiratory cycle) of the patient, wherein the detected biomarker is indicative of a neural respiratory drive (NRD) of the patient.

The detected biomarker may be, e.g., an electromyogram (EMG) signal. In this case, the method may further comprise processing the EMG signal. Processing the EMG signal may, e.g., comprise one of sampling the EMG signal, acquiring an envelope of the EMG signal, and integrating the EMG signal. Processing the EMG signal may, e.g., comprise filtering cardiac artifacts from the EMG signal and/or deriving one or more EMG data values from the EMG signal for each respiratory cycle by obtaining a peak value, and computing a root mean square (RMS) value, a quadratic mean value, or running average value of the EMG signal.

One method further comprises determining a heart rate variability and/or respiration rate variability of the patient from the detected biomarker, and determining a validity of the detected biomarker based on the determined heart rate variability and/or respiration rate variability. The method may further comprise storing the NRD index value if the EMG signal is determined to be valid.

The method further comprises generating an NRD index value based on the detected biomarker. If the biomarker is detected over a plurality of respiratory cycles, generating the NRD index value may further comprise obtaining the highest amplitude, median, or average of the EMG data values over the plurality of respiratory cycles. The method may optionally comprise normalizing the NRD index value. The method may further comprise obtaining a patient-specific reference value, in which case, normalizing the NRD index value comprising computing a function of the NRD index value and the patient-specific reference value.

The method may further comprise graphically displaying the NRD index value to the patient and/or generating an alert or notification signal in response to determining that the onset of the acute exacerbation of the respiratory disease in imminent. If the patient suffers from a respiratory disease (e.g., Chronic Obstructive Pulmonary Disease (COPD)), the method may further comprise determining an extent of the respiratory disease based on the generated NRD index. In this case, the method may further comprise predicting an onset of an acute exacerbation of the respiratory disease in the patient based on the generated NRD index value.

For example, predicting the onset of the acute exacerbation of the respiratory disease in the patient may comprise comparing the NRD index value to an absolute NRD threshold value, and determining that the onset of the acute exacerbation of the respiratory disease is imminent if the NRD index value exceeds the absolute NRD threshold. As another example, predicting the onset of the acute exacerbation of the respiratory disease in the patient may comprise computing an NRD index difference value between the NRD index value and a previously generated NRD index value, comparing the NRD difference value to a differential NRD threshold value, and determining that the onset of the acute exacerbation of the respiratory disease is imminent if the NRD index difference value exceeds the differential NRD threshold value.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a medical monitoring system arranged in accordance with one embodiment of the present inventions;

FIG. 2 is a block diagram of an implantable sensor device used in the medical monitoring system of FIG. 1;

FIG. 3 is a block diagram of an external control unit used in the medical monitoring system of FIG. 1;

FIG. 4 is a flow diagram illustrating one method of operating the medical monitoring system of FIG. 1 to monitor a respiratory disease of a patient;

FIG. 5 is a plan view of an implantation site of the implantable sensor device of FIG. 2 in a second parasternal intercostal muscle of the patient;

FIG. 6a is a diagram of raw EMG data detected by the implantable sensor device of FIG. 2;

FIG. 6b is a diagram of parasternal EMG data derived from the raw EMG data of FIG. 6a;

FIG. 6c is a diagram of ECG data derived from the raw EMG data of FIG. 6a;

FIG. 6d is a diagram of instantaneous heart rate derived from the ECG data of FIG. 6c;

FIG. 6e is a diagram of an envelope EMG derived from the parasternal EMG data of FIG. 6b;

FIG. 6f is a diagram of an instantaneous respiration rate derived from the envelope EMG of FIG. 6e;

FIG. 6g is a diagram of peak EMG values derived from the envelope EMG of FIG. 6e; and

FIG. 6h is a diagram of normalized neural respiratory drive index values derived from the instantaneous respiration rate of FIG. 6f and the peak EMG data values of FIG. 6g;

FIG. 7 is a flow diagram illustrating another method of operating the medical monitoring system of FIG. 1 to monitor a respiratory disease of a patient; and

FIG. 8 is a plan view of a medical monitoring system arranged in accordance with another embodiment of the present inventions.

### DETAILED DESCRIPTION OF THE EMBODIMENTS

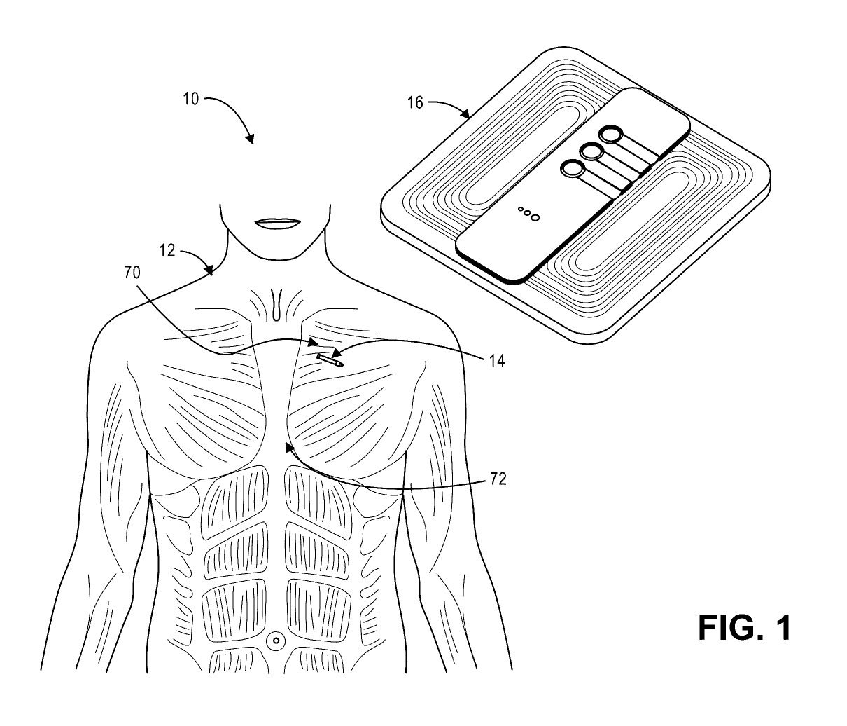

Turning first to FIG. 1, an exemplary medical monitoring system 10 constructed in accordance with the present inventions will now be described. The medical monitoring system 10 is configured for indirectly assessing a neural respiratory drive (NRD) of a patient 12 suffering from a respiratory disease (e.g., Chronic Obstructive Pulmonary Disease (COPD)) by monitoring aspects of the respiration of the patient 12 over one or more respiratory cycles. For example, the medical monitoring system 10 may track the NRD of the patient 12 by sensing biomarkers indicative of the NRD (e.g., electromyogram (EMG) signals), and generating a neural respiratory drive (NRD) index (a value representative of the NRD) based on the sensed biomarkers. The medical monitoring system 10 is further configured for predicting an onset of an acute exacerbation of the respiratory disease in the patient 12 based on the generated NRD index value, and alerting/notifying the patient 12 (or attending caretaker) if the onset of the acute exacerbation of the respiratory disease is imminent, requiring proactive measures to prevent hospitalization. Alternatively, or in addition to monitoring the onset of the acute exacerbation of a respiratory disease, the medical monitoring system 10 monitor the recovery of the patient 12 from the acute exacerbation of the respiratory disease or in response to different treatments of the respiratory disease.

To this end, the medical monitoring system 10 generally comprises an implantable sensor device 14 configured for being implanted within and sensing EMG signals in the patient 12, and an external control unit 16 device configured for transcutaneously controlling and providing power to the sensor device 14 to sense the EMG signals, transcutaneously receiving EMG signals from the sensor device 14, deriving resting NRD index values from the EMG signals, storing and displaying the resting NRD index values to the patient 12, analyzing the resting NRD index values to predict the onset of an acute COPD exacerbation in the patient 12, and if necessary, notifying/alerting the patient 12 that acute COPD exacerbation in the patient 12 is imminent.

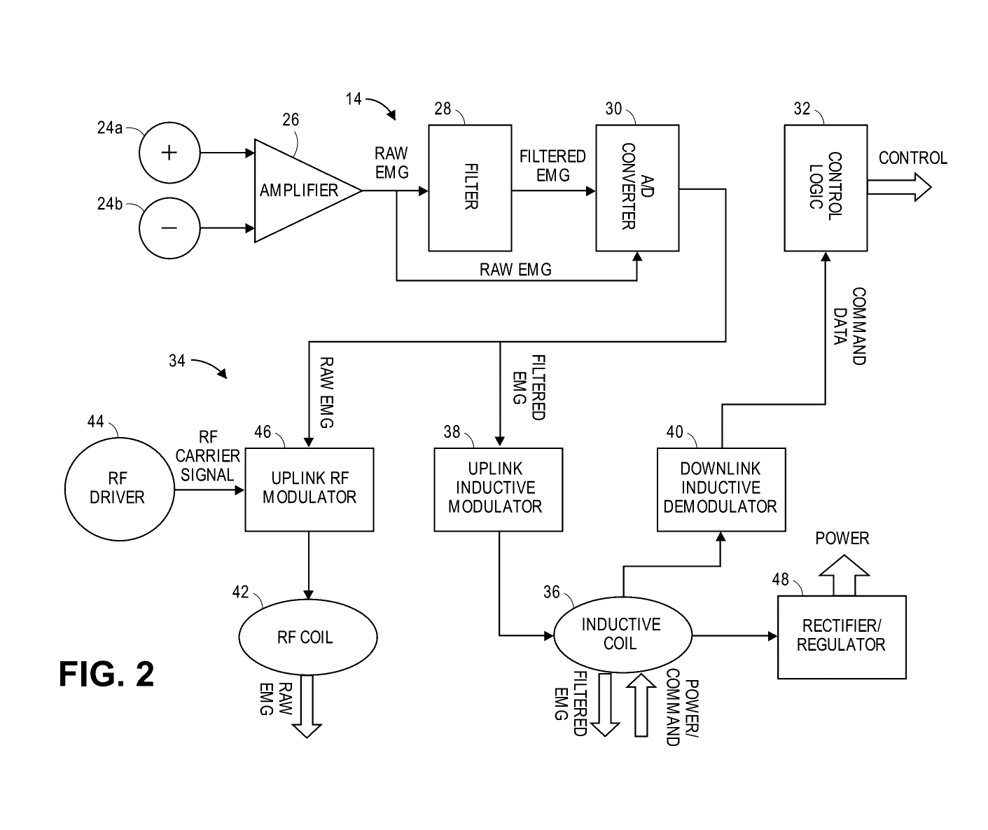

Referring further to FIG. 2, the sensor device 14 comprises two differential recording electrodes 24a, 24b configured for sensing electrical activity within the muscle fibers in which the sensor device 14 is implanted and outputting a raw analog EMG signal; one or more adjustable gain amplifiers 26 configured for amplifying the EMG signal; a filter 28 configured for obtaining an envelope, integrating, or sampling the EMG signal; an analog-to-digital converter (ND) converter 30 configured for selectively transforming either the raw EMG signal output from the amplifier(s) 26 or the filtered EMG signal output from the filter 28 into a digitized EMG signal; and control logic 32 (e.g., command processor, frame generator, PLL logic, command decoder, and error correction circuitry) configured for controlling and operating the sensor device 14 in accordance with commands received from the external control unit 16. The filter 28 can also be realized digitally. In this case, the filter 28 will be placed after the ND converter 30.

The sensor device 14 further comprises telemetry/power circuitry 34 configured for receiving commands and power from the external control unit 16 and transmitting the EMG signal (either raw or filtered) to the external control unit 16. In the illustrated embodiment, the sensor device 14 utilizes a robust half-duplex low-bandwidth data link for transmitting the filtered EMG signal to the external control unit 16 and receiving command data from the external control unit 16, and a high-bandwidth data link for transmitting the EMG signal to the external control unit 16.

To this end, the telemetry/power circuitry 34 may comprise an inductive coil 36 configured for receiving an alternating current (AC) power signal inductively transmitted from the external control unit 16. The telemetry/power circuitry 34 utilizes the AC power signal as both a source of power and as a low-bandwidth downlink/uplink carrier signal. In particular, the telemetry/power circuitry 34 further comprises an uplink inductive modulator 38 configured for modulating the inductive carrier signal with the filtered EMG signal received from the A/D converter 30 by varying the inductive carrier signal at the inductive coil 36 in accordance with the filtered EMG signal. In the illustrated embodiment, the uplink inductive modulator 38 is a load modulator that amplitude modulates the inductive carrier signal by varying the impedance of the inductive coil 36, which as will be described below, allows the external control unit 16 to acquire the filtered EMG signal from the inductive carrier signal. The telemetry/power circuitry 34 further comprises a downlink inductive demodulator 40 configured for demodulating command data received from the external control unit 16 from the inductive carrier signal at the inductive coil 36. In the illustrated embodiment, the downlink inductive demodulator 40 is an amplitude modulation (AM) demodulator that demodulates the downlink data by measuring the amplitude variations of the AC power signal. Although a single inductive coil 36 is described as being used for both power reception and EMG data transmission, in alternative embodiments, separate dedicated coils can be respectively used for power reception and EMG data transmission.

The telemetry/power circuitry 34 further comprises a radio frequency (RF) coil 42 and a radio frequency (RF) driver 44 in the form of an oscillator for applying an RF carrier signal to the RF coil 42. The telemetry/power circuitry 34 utilizes the RF signal as a high-bandwidth uplink carrier signal. In particular, the telemetry/power circuitry 34 further comprise uplink RF modulator 46 configured for modulating the RF carrier signal with the raw EMG signal received from the A/D converter 30, thereby allowing the external control unit 16 to acquire the raw EMG signal from the RF carrier signal.

The telemetry/power circuitry 34 further comprises a rectifier/regulator 48 for rectifying and regulating the inductive carrier signal received at the inductive coil 36 for powering the circuitry of the sensor device 14. In alternative embodiments, the sensor device 14 may include a rechargeable battery (not shown) for storing the electrical energy, or a non-rechargeable battery, in which case, power may be supplied to the circuitry of the sensor device 14 without connection to the external control unit 16. In this case, the sensor device 14 may further comprise memory (not shown) for storing the EMG signal that can be subsequently transmitted via a dedicated communication coil upon interrogation of the sensor device 14 by the external control unit 16. In optional embodiments, status signals can be transmitted by the sensor device 14 to the external control unit 16 via coil 36 or coil 42 to provide, for example, battery status information or other operational information of the sensor device 14.

The sensor device 14 may take the form of a miniaturizing cylindrical sensing device, with the circuitry being implemented as a sub-assembly on a single-chip integrated circuit mounted on a ceramic substrate sandwiched between two halves of a cylindrical magnetic core around which the RF and inductive coils are wound. The electronics are encapsulated in a cylindrical ceramic package that include two metal endcaps at opposite ends of the ceramic package that serve as the differential recording electrodes. Such an implantable sensor device allows the EMG signals to be detected at the implantation site of this device. A commercial example of such an implantable sensor device 14 is the IMES® device manufactured by Alfred Mann Foundation and described in Implantable Myoelectric Sensors (IMESs) for Intramuscular Electromyogram Recording, IEEE Trans Biomed Eng. 2009 January, pp. 159-171. In an alternative embodiment, the sensor device 14 may include a lead (not shown) on which the electrodes are carried, so that EMG signals can be detected at a location remote from the implantation site of the body of the device.

It should be appreciated that although a specific embodiment of the sensor device 14 has been described herein, the sensor device 14 can take the form of any sensor device sized to be implantable within the patient 12 for acquiring EMG signals. For example, the sensor device 14 may be any one of the microsensors disclosed in U.S. Pat. Nos. 6,185,452, 6,164,284, 6,564,807, 8,684,009, 8,555,894, 7,513,257, 6,315,721, 6,208,894, 6,067,474, and 7,114,502, which are expressly incorporated herein by reference.

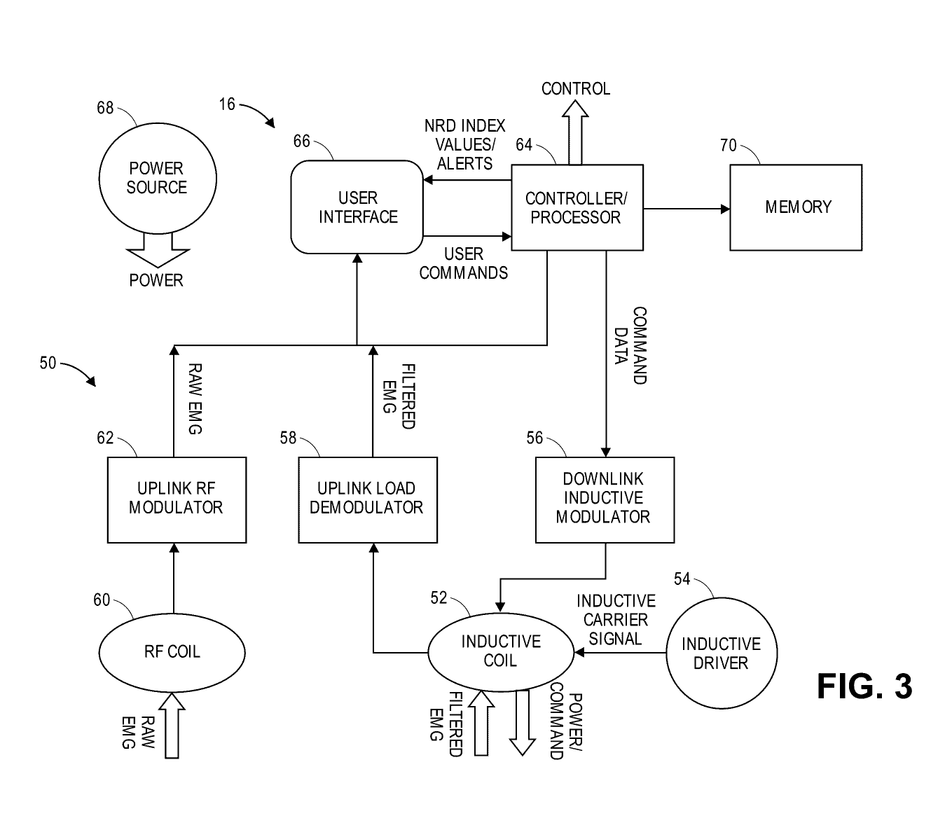

Referring further to FIG. 3, the external control unit 16 comprises telemetry/power circuitry 50 configured for transmitting commands and power to the sensor device 14 and receiving the EMG signal (either raw or filtered) from the sensor device 14. As discussed above, low-bandwidth data link is utilized for receiving the filtered EMG signal from the sensor device 14 and transmitting command data to the sensor device 14, and the high-bandwidth data link is utilized for receiving the raw EMG signal from the sensor device 14.

To this end, the telemetry/power circuitry 50 comprises an inductive coil 52 configured for inductively transmitting the afore-mentioned AC power signal to the sensor device 14. As described above, the AC power signal is utilized as both a source of power and as a low-bandwidth downlink/uplink carrier signal. To this end, the telemetry/power circuitry 50 further comprises a downlink inductive modulator 56 configured for modulating the inductive carrier signal with command data by varying the inductive carrier signal at the inductive coil 52 in accordance with the command data. Like the uplink inductive modulator 38 in the sensor device 14. The downlink inductive modulator 56 is an amplitude modulator that modulates the amplitude of the inductive carrier signal, thereby allowing the sensor device 14 to acquire the command data as described above. The telemetry/power circuitry 50 further comprises an uplink inductive demodulator 58 configured for demodulating filtered EMG data received from the sensor device 14 from the inductive carrier signal at the inductive coil 52. In the illustrated embodiment, the uplink inductive demodulator 58 is a load demodulator that demodulates the inductive carrier signal by measuring the AC amplitude variations of the inductive coil 52. Although a single inductive coil 52 is described as being used for both power transmission and EMG data reception, in alternative embodiments, separate dedicated coils can be respectively used for power transmission and EMG data reception. The telemetry/power circuitry 50 further comprises an RF coil 60 and an uplink RF demodulator 62 configured for demodulating the RF carrier signal to obtain the raw EMG signal from the sensor device 14.

The external control unit 16 further comprising a controller/processor 64 configured for controlling and operating the external control unit 16, processing the EMG signal (raw or filtered) received from the sensor device 14, deriving the resting NRD index value, and predicting an onset of an acute exacerbation of the respiratory disease in the patient 12 based on the generated NRD index value, and alerting/notifying the patient 12 (or attending caretaker) if the onset of the acute exacerbation of the respiratory disease is imminent. In an alternative embodiment, the controller/processor 64 may be configured for acquiring patient-specific reference values, which may be derived from the EMG signal, and normalizing the resting NRD index value based on patient-specific reference values. All of these functions will be described in further detail below.

The external control unit 16 further comprises a user interface 66 configured for receiving input from a user, e.g., via buttons, and for graphically displaying NRD index values, providing alerts/notifications, and for querying the patient 12 (or attending caretaker), e.g., via a display. The controller/processor 64 is further configured for, in response to input into the user interface 66, generating the commands that are transmitted to the sensor device 14 via the telemetry/power circuitry 50 to acquire the EMG signals from the sensor device 14. For example, the user interface 66 may have a button that can be actuated to send power and command signals to the sensor device 14 to measure and return the EMG signal to the external control unit 16. The external control unit 16 further comprises a power source 68, e.g., a battery, for providing power to the circuitry of the external control unit 16, and memory 70 configured for storing information, such as NRD index values and patient-specific reference values.

Although the external control unit 16 is described herein as processing the EMG signals to obtain NRD index values, it should be appreciated that the sensor device 14 can alternatively function to process the EMG signals to obtain NRD index values for transmission to the external control unit 16. Furthermore, although the external control unit 16 is described herein as predicting the onset of an acute COPD exacerbation in the patient 12, it should be appreciated that the sensor device 14 can alternatively function to predict the onset of an acute COPD exacerbation in the patient 12, and send any alerts/notifications to the external control unit 16 for display to the patient 12, or may event directly alert/notify the patient 12 via other means, e.g., vibrations.

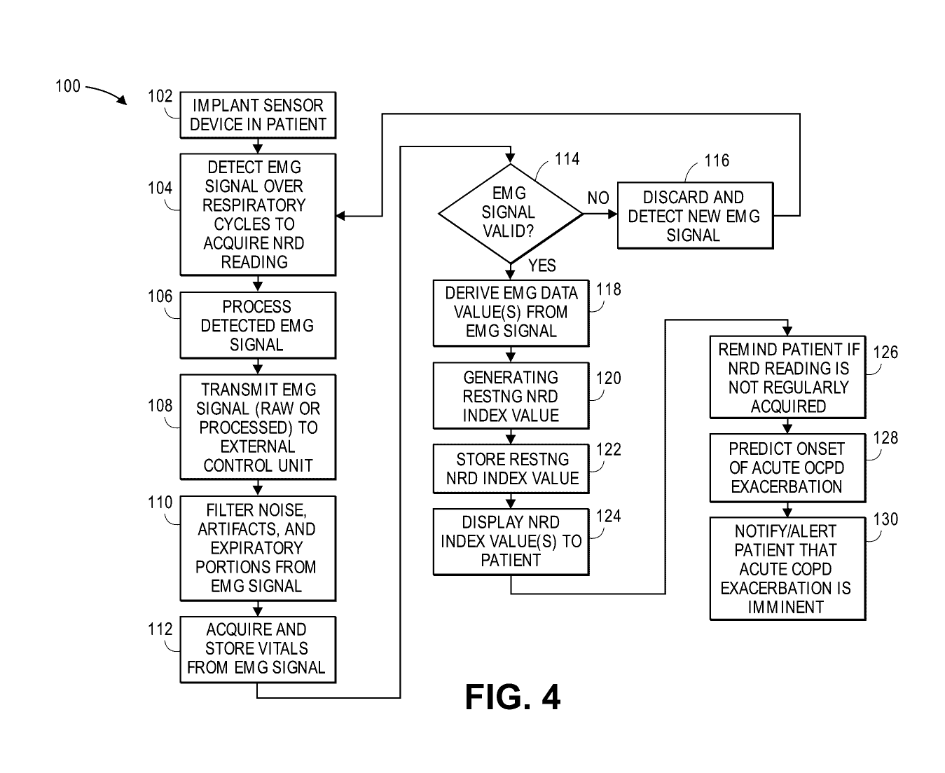

Having described the function and arrangement of the medical monitoring system 10, one method 100 of operating the medical monitoring system 10 to monitor the NRD, and thus the COPD status, of the patient 12 during several respiratory cycles will now be described with reference to FIG. 4.

As an initial matter, it should be noted that the respiratory cycle consists of alternating processes of inspiration and expiration. During inspiration, skeletal muscles, such as the diaphragm and external intercostal muscles contract and allow for expansion of the thoracic cavity. As the volume of the pleural space increases, the pressure in this area drops, and with it, the pressure inside the lung (intrapulmonary pressure) also drops. The pressure gradient allows for rapid air flow into the lung and inspiration occurs. During expiration, the inspiratory muscles relax causing the volume of the thoracic cavity to be reduced. The relaxation of the diaphragm increases intrapleural and intrapulmonary pressures, forcing the gases in the lungs out. Normally, unlabored expiration at rest is passive by relaxation of inspiratory muscles. When an increase in pulmonary ventilation is required, such as during exercise, expiration occurs actively dependent upon contraction of accessory, expiratory muscles that pull down the rib cage and compress the lungs.

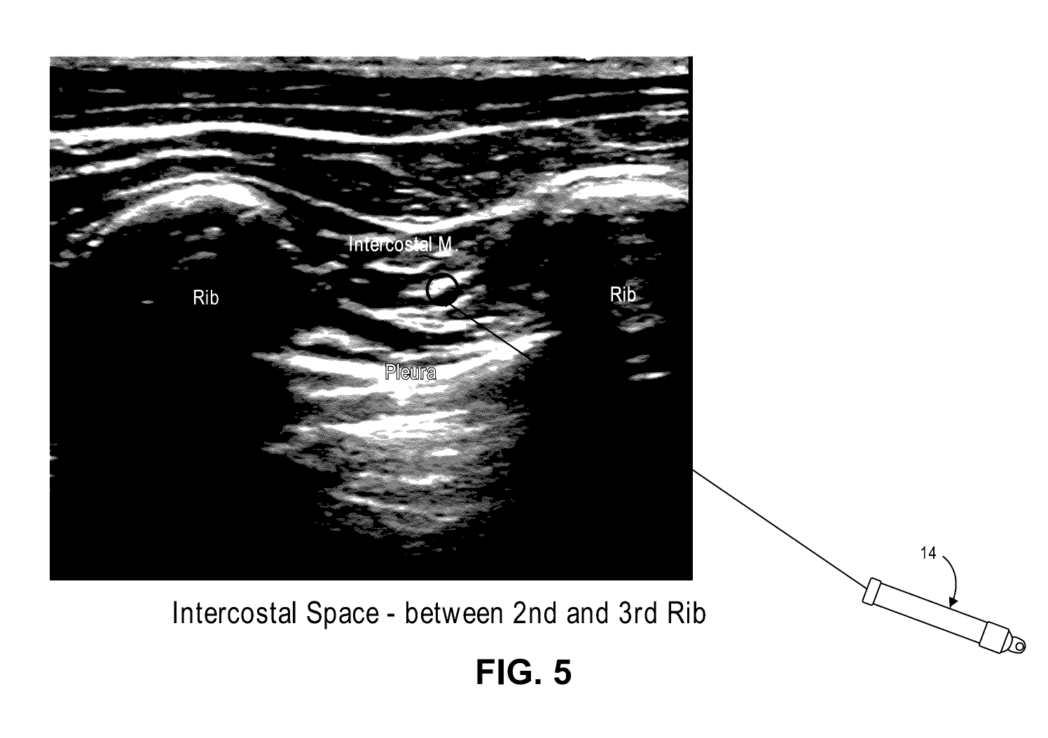

First, the sensor device 14 is implanted within a muscle that is activated during respiration of the patient 12 (step 102). In the illustrated embodiment, the sensor device 14 is implanted within the intercostal muscle of the patient 12, such as one of the 2^{nd} parasternal intercostal muscles 70, as illustrated in FIG. 5. Although the sensor device 14 is shown implanted on the left chest area of the patient 12, it should be appreciated that the sensor device 14 may alternatively be implanted on the right chest area of the patient 12. Alternatively, the sensor device 14 may be implanted within the diaphragm 72 of the patient 12. The sensor device 14 may be implanted within the patient 12 using an implantation guide or guide wire configured to be visible using ultrasound, allowing the implantation procedure to be guided using ultrasound imaging.

Next, an EMG signal is detected by the implanted sensor device 14 during several respiratory cycles of the patient 12 to obtain an NRD reading (step 104). This can be accomplished by placing the external control unit 16 into operative communication with the implanted sensor device 12; for example, by placing the external control unit 16 over the implanted sensor device 14 and operating it to send command signals to the implanted sensor device 14 to detect the EMG signal. For example, the patient 12 (or attending caretaker) may place the external control unit 16 over the implanted sensor device 14 for a period, e.g., between thirty seconds and three minutes, during which the patient 12 is breathing in a relaxed manner, such that the EMG signal is detected during a resting respiratory effort of the patient 12. The implanted sensor device 14 then processes the EMG signal (e.g., by sampling, generating an envelope, or integrating) (step 106), and wirelessly transmits the EMG signal (either raw or processed) to the external control unit 16 (step 108).

The external control unit 16 then filters noise and artifacts out of the EMG signal to be a better representative of the respiratory effort of the patient 12 (step 110). For example, cardiac artifacts, such as QRS complexes, may appear in the EMG signal, in which case, the external control unit 16 may filter the QRS complexes from the EMG signal. In one embodiment, the external control unit 16 further processes the EMG signal to be a better representative of the inspiratory effort of the patient 12. For example, the external control unit 16 may filter out the expiratory portions of EMG signal, so that only the portion of the EMG signal corresponding to the inspiratory effort of the EMG signal remains.

The external control unit 16 then acquires vitals (such as heart rate and/or respiration rate) of the patient 12 from the EMG signal (step 112), and stores values representing the average heart rate, heart rate variability, average respiration rate, and/or respiration rate variability. The QRS complexes filtered from the EMG signal at step 110 may be used to determine the mean heart rate or heart variability of the patient. Furthermore, the natural cycles of the EMG signal can be used to determine the mean respiration rate or respiration variability.

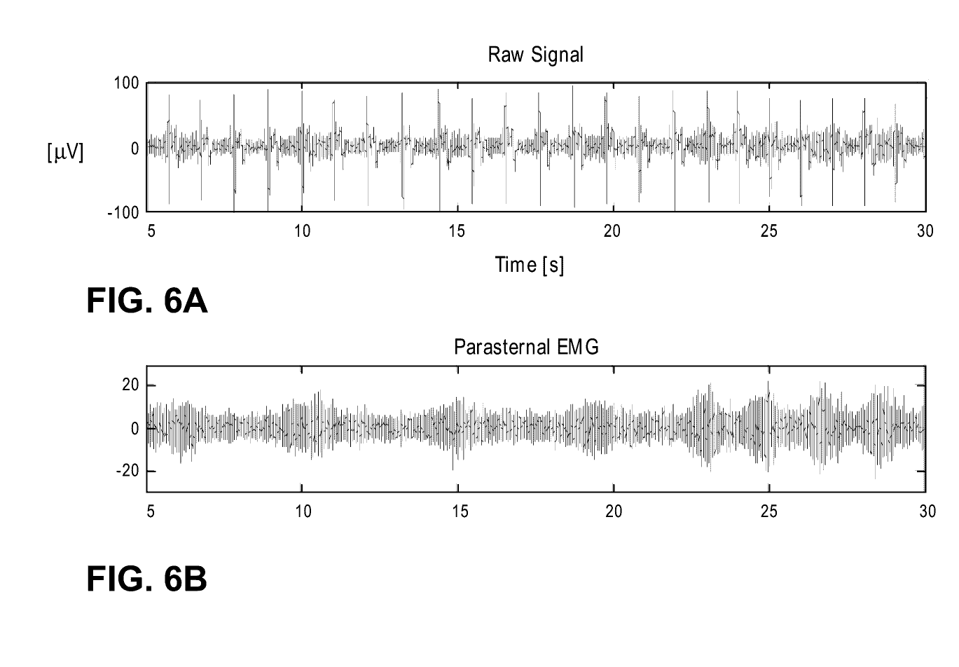

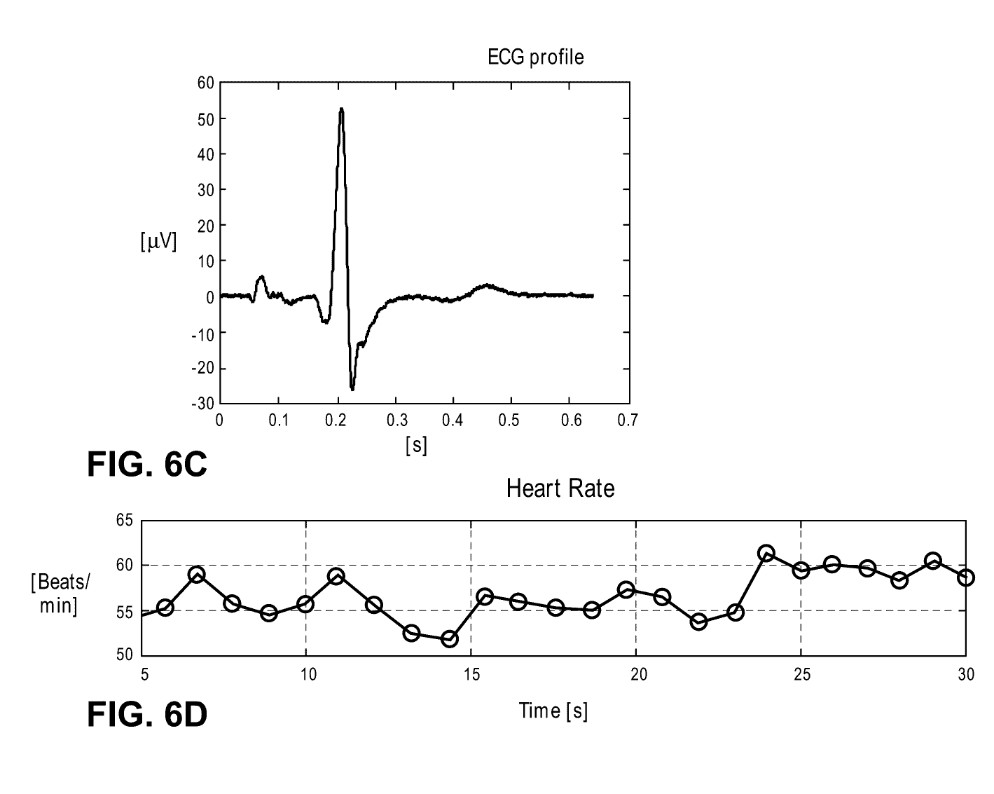

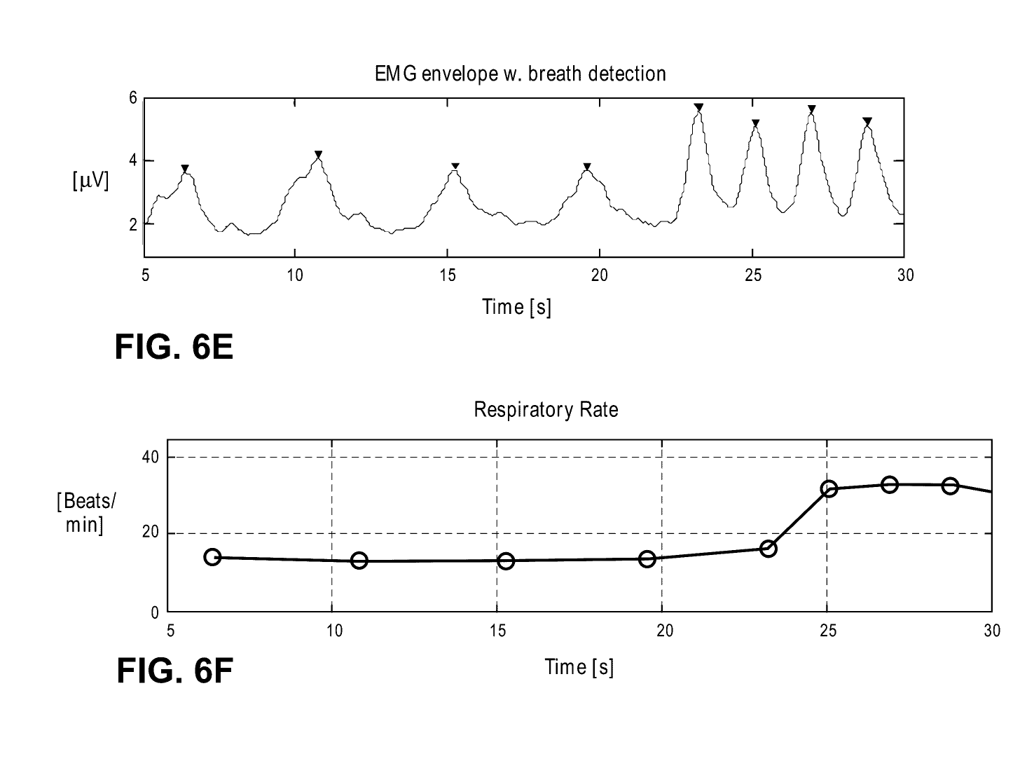

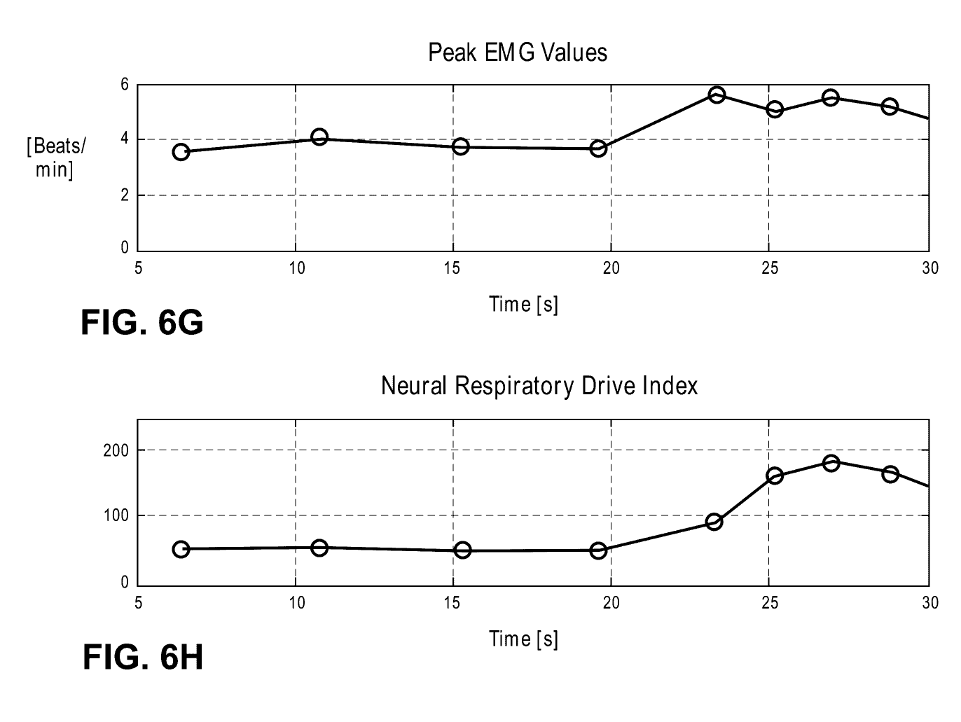

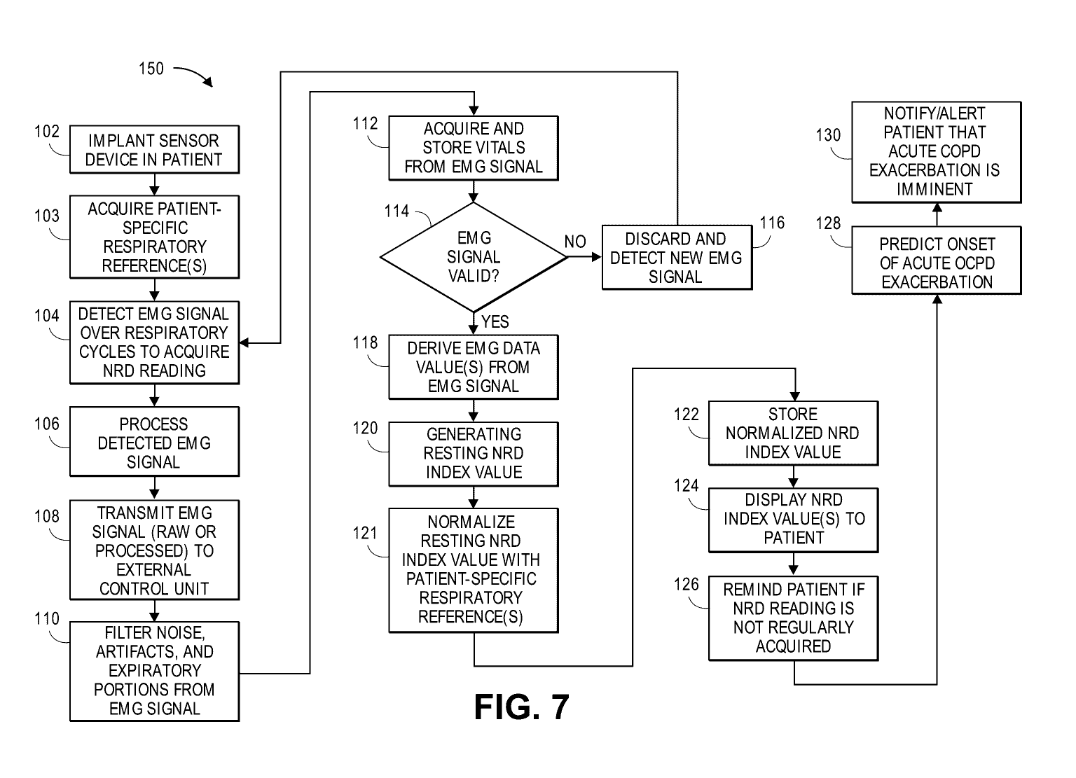

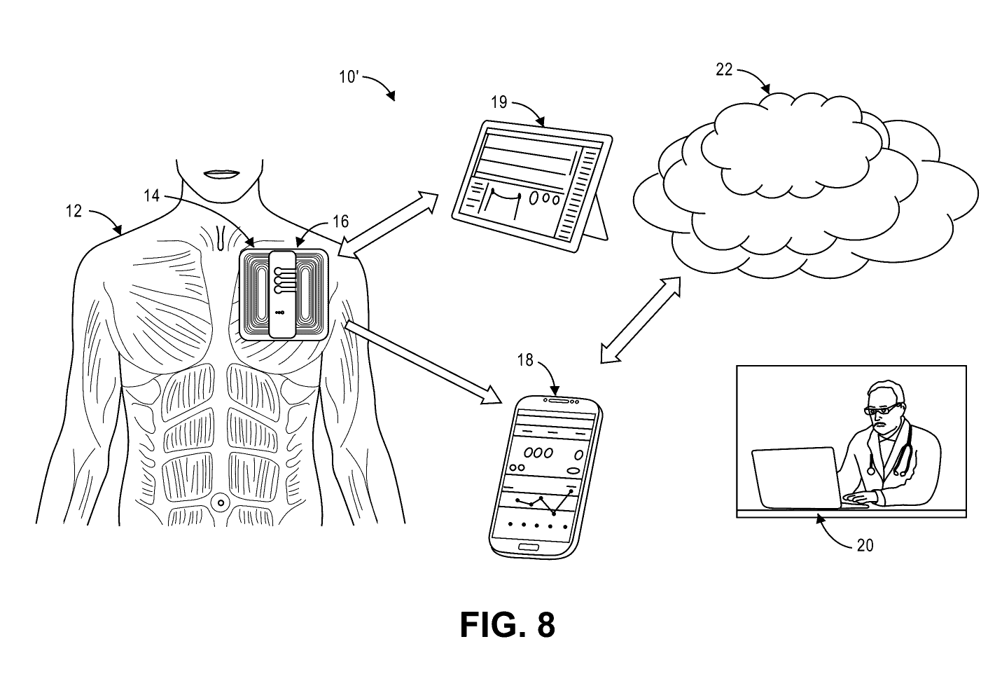

For example, an average electrocardiogram (ECG) may be derived and subtracted from a raw EMG signal (FIG. 6a), resulting in a purely respiratory EMG signal (parasternal EMG) (FIG. 6b) and an average ECG profile (FIG. 6c). The instantaneous heart rate (beats/minute) (FIG. 6d) can be derived from the average ECG profile. The average heart rate and/or heart rate variability can then be computed from the instantaneous heart rate. The parasternal EMG can be processed to obtain an EMG envelope (FIG. 6e), the peaks of which can be identified to determine the instantaneous respiratory rate (breaths/minute) (FIG. 6f). The average respiratory rate and/or respiratory rate variability can then be computed from the instantaneous respiratory rate. The values representing the average heart rate, heart rate variability, average respiration rate, and/or respiration rate variability can be used as indicators of the general wellness of the patient 12, and as will be described in further detail below, may be used to normalize the NRD reading of the patient 12.

Next, the external control unit 16 determines if the EMG signal is valid (step 114). For example, it is preferred that the patient 12 remains in a state of relaxed breathing while an NRD reading is acquired over multiple respiratory cycles. A high respiration rate and/or peak breath amplitude variability may indicate that the patient 12 is anxious or otherwise did not remain in a state of relaxed breathing, and that the resting EMG signal is therefore unreliable. Thus, the external control unit 16 may use the respiration rate variability and/or peak breath amplitude variability during an NRD reading, which can be acquired from the resting EMG signal, to evaluate the quality of the resting EMG signal.

Alternatively, or in addition to, the automatic evaluation of the quality of the current EMG by the external control unit 16, the external control unit 16 may query the patient 12 (or attending caretaker) whether the EMG signal is valid. For example, the external control unit can have one or more buttons that can be actuated, which allows the patient 12 (or attending caretaker) to selectively discard the EMG signal or store the EMG signal. This may be useful if the patient 12 (or attending caretaker) encounters problems during a NRD reading, such as loss of power to the external control unit 16, loss of connection between the external control unit 16 and the implantable sensor device 14, or if the patient 12 stops breathing in a relaxed manner during the NRD reading.

If the EMG signal is determined to be invalid, the external control unit 16 discards the EMG signal and commands the implanted sensor device 14 to acquire another EMG signal (step 116). If the EMG signal is determined to be valid, the external control unit 16 generates the resting NRD index value.

In particular, the external control unit 16 may further derive one or more EMG data values from the EMG signal for each respiratory cycle to facilitate subsequent computation of the resting NRD index value of the patient 12 (step 118). For example, the external control unit 16 may obtain a peak value, compute a root mean square (RMS) value, a quadratic mean value, or running average value of the EMG signal for each respiratory cycle.

Next, the external control unit 16 generates the resting NRD index value based on the EMG data values (step 120). For example, the external control unit 16 may obtain the highest peak amplitude of the processed EMG data values over the respiratory cycles to obtain a single value representing the highest measured respiratory effort of the patient 12 or compute a mean or average of the peak amplitudes of the processed EMG data values over the respiratory cycles to obtain a single value representing the average or mean measured respiratory effort of the patient 12. The highest, average, or mean of the measured respiratory effort may be used as the current respiratory effort of the patient 12, and therefore, represents the resting NRD index value. For example, peak EMG data values (FIG. 6g), one for each respiratory cycle (breath), may be acquired from the EMG envelope (FIG. 6e). The NRD index value may, e.g., the highest EMG data value of approximately 5.8, or may be, e.g., an average of the EMG data values, approximately 4.5.

Next, the external control unit 16 stores the resting NRD index value (step 122), and graphically displays the resting NRD index value, along with any other previously generated NRD index values, to the patient 12 (or attending caretaker) over time (step 124). The external control unit 16 may optionally remind the patient 12 (or attending caretaker) if NRD reading of the patient 12 is not being regularly performed (step 126). For example, if daily readings of the NRD of the patient 12 are desired, the external control unit 16 may alert the patient 12 (or attending caretaker) that a reading of the NRD should take place.

It should be appreciated that at any point in the method 100, steps 104-126 may be repeated to generate additional resting NRD index values.

Next, the external control unit 16 predicts the onset of the acute COPD exacerbation in the patient 12 based on the resting NRD index value (step 128). It is known that in patients with COPD, an increase in NRD may indicate that an acute COPD exacerbation is about to occur. That is, if the NRD index (and thus, the NRD) rises by a certain percentage, or undergoes a certain rate of change or acceleration, it may be determined that the acute COPD exacerbation is imminent (e.g., will happen the same day, tomorrow, or within a week). For example, the external control unit 16 may compare an increase percentage in the NRD based on the resting NRD index value and one or more previously generated NRD index values, and predict that the onset of the acute COPD exacerbation is imminent if the NRD increase is great enough.

In one embodiment, the external control unit 16 computes the percentage difference between resting NRD index value and the immediate previously generated NRD index value to obtain an NRD index difference value, which is then compared to a NRD index difference threshold value. If the NRD index difference value exceeds the NRD index difference threshold value, the external control unit 16 determines that the onset of the acute COPD exacerbation is imminent. For example, assume the resting NRD index value is 1000, and the previous NRD index value is 600. The NRD index difference value (i.e., the percentage change in NRD) would be 67%. If the NRD index difference threshold value is 50%, the NRD index difference value of 67% would exceed this threshold value, and therefore, the external control unit 16 will predict that the onset of the acute COPD exacerbation in the patient 12 is imminent.

In another embodiment, the external control unit 16 computes the percentage rate of change with respect to time between the resting NRD index value and the immediate previously generated NRD index value to obtain an NRD index rate of change value, which is then compared to an NRD index rate of change threshold value. If the NRD index rate of change value exceeds the NRD index rate of change threshold value, the external control unit 16 determines that the onset of the acute COPD exacerbation is imminent. For example, assume the resting NRD index value is 1000, and the previous NRD index value is 800, and these NRD readings are taken 12 hours apart. The NRD index rate of change value (i.e., the percentage rate of change in NRD) would be 50%/day. If the NRD index rate of change threshold value is 30%, the NRD index rate of change value of 50% would exceed this threshold value, and therefore, the external control unit 16 will predict that the onset of the acute COPD exacerbation in the patient 12 is imminent.

In still another embodiment, the external control unit 16 computes the percentage rate of acceleration with respect to time of a series of NRD index values to obtain an NRD index rate of acceleration value, which is then compared to an NRD index rate of acceleration threshold value. If the NRD index rate of acceleration value exceeds the NRD index rate of acceleration threshold value, the external control unit 16 determines that the onset of the acute COPD exacerbation is imminent. For example, assume the resting NRD index value is 1000, the first previous NRD index value is 900, and the second previous NRD index value is 820. The NRD index rate of acceleration value (the rate of acceleration in NRD) would be 25%/day. If the NRD index rate of acceleration threshold value is 10%, the NRD index rate of acceleration value of 25% would exceed this threshold value, and therefore, the external control unit 16 will predict that the onset of the acute COPD exacerbation in the patient 12 is imminent.

Lastly, if the external control unit 16 predicts that the onset of the acute COPD exacerbation in the patient 12, the external control unit 16 will notify/alert the patient 12 (or attending caretaker) that acute COPD exacerbation in the patient 12 is imminent, and that the patient 12 should be admitted to a hospital or otherwise examined by a physician (step 130). If the external control unit 16 predicts that the onset of the acute COPD exacerbation in the patient 12 is not imminent, the process returns to step 104 to take another NRD reading at a future time.

It should be noted that in previously available methods for measuring the NRD of the patient 12, measurement of the patient's current respiratory effort was not consistently accomplishable from reading-to-reading without normalizing the current respiratory effort against the maximum respiratory effort. Accordingly, both current respiratory effort and maximum respiratory effort has to be measured every time an NRD reading was desired, and compared to each other to obtain a percentage NRD index value. In contrast, the method 100 described above may accurately measure the NRD of the patient 12 without normalization, since the implantation of the sensor device 14 allows the recording electrodes to consistently take NRD readings without concern that any changes in the NRD readings will be caused by any factor other than a change in the condition of the COPD of the patient 12. Furthermore, because the method 100 compares NRD index values to each other in predicting the acute exacerbation of the patient 12, any variations from patient-to-patient can be ignored.

In an alternative embodiment, the external control unit 16 may normalize the NRD readings in order to acquire NRD index values that can be meaningfully compared from patient-to-patient. For example, with reference to FIG. 7, another method 150 of operating the medical monitoring system 10 to monitor the NRD, and thus the COPD status, of the patient 12 during several respiratory cycles will now be described. The method 150 is similar to the method 100, with the exception that method 150 generates normalized NRD index values to the patient 12.

After the sensor device 14 is implanted at step 102, one or more patient-specific respiratory references are acquired from the patient 12 and stored by the external control unit 16 (step 103). In the illustrated embodiment, a patient-specific respiratory reference is obtained by detecting EMG signal during a maximum respiratory effort of the patient 12, and generating a maximum NRD index from EMG signal. The maximum respiratory effort represents the highest respiratory effort that the patient 12 is capable of, and can be calibrated at step 104 by asking the patient 12 to perform a series of maximum “sniff” maneuvers and detecting an EMG signal to find a maximum achieved value (representative of the maximum NRD index value of the patient 12). The EMG signals during the maximum respiratory effort of the patient 12 may be acquired and processed in the same manner as the resting EMG signals are acquired and processed during the resting respiratory effort of the patient 12 at steps 104-110 in the method 100, and the maximum NRD index can be generated and stored in the same manner as the resting NRD index in steps 118-122 in the method 100.

After the resting EMG signal is acquired and the NRD index is generated at steps 104-120, the external control unit 16 normalizes the resting NRD index value to the patient 12 by computing a function of the resting NRD index value obtained at step 120 and the patient-specific respiratory reference(s) obtained at step 103, and in this case, the maximum NRD index value (step 121). To normalize the resting NRD index value, the resting NRD index value can be divided by the maximum NRD index value to obtain a percentage NRD index value, which may represent the completely normalized NRD index value. Alternatively, instead directly normalizing the resting NRD index value with the patient-specific respiratory reference(s) obtained at step 103, the EMG data values derived at step 118 can be normalized with the patient-specific respiratory reference(s) to acquire normalized EMG data values, and then a normalized NRD index value can be derived from the normalized EMG data values.

Next, the external control unit 16 predicts the onset of the acute COPD exacerbation in the patient 12 based on the normalized resting NRD index value (step 128). It is known that in patients with COPD, an NRD that reaches a certain value may indicate that an acute COPD exacerbation is about to occur. Because the NRD index value is normalized, it need not be compared to previous NRD index values to obtain an accurate indication of whether the acute COPD exacerbation is imminent (e.g., will happen the same day, tomorrow, or within a week). Rather, the normalized NRD index value can just be compared to an absolute NRD index threshold value, that can be the same from patient-to-patient. For example, assume the normalized resting NRD index value is 50, and the absolute NRD index threshold value is 40. Regardless of the previous resting NRD index values, the external control unit 16 determines that the onset of the acute COPD exacerbation is imminent, since the normalized resting NRD index value is greater than the absolute NRD index threshold value.

Although, in previously available methods for measuring the NRD of the patient 12, the maximum respiratory effort had to be calibrated each time the resting NRD index value is to be normalized, e.g., by requesting the patient 12 to perform a series of “sniff” maneuvers just prior to obtaining each NRD index value from the patient 12, the maximum respiratory effort need only be calibrated a single time due to the stability of the medical monitoring system 10. That is, the implanted sensor device 14 will not move or migrate from the implantation site, and thus, it is expected that the maximum respiratory effort of the patient 12 will not change over time. In alternative embodiments, the maximum respiratory effort is calibrated periodically, but not every time the current resting NRD index value is acquired.

In an alternative embodiment, another patient-specific respiratory reference may be detected by detecting a respiratory rate of the patient 12, which can be acquired from the natural respiratory periods of the EMG signal at step 108. The respiratory rate of the patient 12 may be a significant normalizing factor, since a patient 12 may take many shallow breaths, as opposed to taking fewer deeper breaths, while expending the same amount of respiratory effort, yet the unnormalized NRD index value for the patient 12 when taking shallow breaths will be less than the NRD index value for the patient 12 when taking deeper breaths. As such, the un-normalized NRD index value generated at step 120 or the normalized NRD index generated at step 121 can be multiplying by the respiratory rate of the patient 12 to obtain a completely normalized NRD index value. Alternatively, the peak EMG data values (FIG. 6g) may be normalized by respectively multiplying them by the respiratory rate values (FIG. 6f) to obtain a normalized NRD index value for each breath (FIG. 6h). A single normalized NRD index value (highest or average) can then be derived from the NRD index values.

In another alternative embodiment, still another patient-specific respiratory reference may be detected measuring one of a maximum volume, force expiratory volume (FEV), and forced vital capacity (FVC), e.g., by using a spirometer. In this case, the un-normalized NRD index value generated at step 120 or the normalized NRD index generated at step 121 can be further normalized by dividing it by the maximum volume, FEV, or FVC to obtain a completely normalized NRD index value. Further normalization of the NRD index value may be performed based on the height, weight, body mass index, age, or race of the patient 12. These patient-specific respiratory references are not expected to substantially change over time, they only need to be acquired once.

Although the external control device 16 has been described as performing the functions of ECG signal processing, NRD index value generation and display, and acute COPD exacerbation onset prediction, these functions can be performed by other external components.

For example, with reference to FIG. 7, another medical monitoring system 10′ constructed in accordance with the present inventions is similar to the medical monitoring system 10 of FIG. 1 with the exception that the medical monitoring system 10′ further comprises a personal wireless device 18 configured receiving the resting NRD index values from the external control unit 16, and storing and displaying the resting NRD index values to the patient 12. The personal wireless device 18 may, e.g., take the form of a conventional Smartphone configured with a smartphone application that stores, tracks, and/or displays the resting NRD index values. In this case, the external control unit 16 further comprises a communications interface (e.g., a USB serial interface or Bluetooth) (not shown) configured for receiving commands from the personal wireless device 18 and transmitting the resting NRD index values to the personal wireless device 18 in response to the commands. The medical monitoring system 10′ may further comprise a patient programmer 19 configured for being operating by a clinician to program the sensor device 14 and/or the external control device 16, as well as for downloading EMG data, patient-specific references, and/or NRD index values from the sensor device 14 and/or external control device 16.

The medical monitoring system 10′ further comprises a remote computer 20 configured for receiving the resting NRD index values and any patient-specific reference values if necessary for normalization of the NRD index values, from the personal wireless device 18 over a wireless network 22, and analyzing the resting NRD index values to predict the onset of an acute COPD exacerbation in the patient 12, and if necessary, notifying/alerting the patient 12 via the personal wireless device 18 over the wireless network 22 that acute COPD exacerbation in the patient 12 is imminent. The remote computer 20 may, e.g., take the form of any conventional personal computer (PC) configured with software capable of predicting the onset of an acute COPD exacerbation of the patient 12. The remote computer 20 may use a cloud computing architecture. The wireless network 22 may, e.g., comprise a wireless local area computing network (e.g., through “Wi-Fi”) or a mobile telecommunication data transfer system (e.g., systems using the 4G standard). The remote computer 20 may be operated by a patient care provider 23 that may analyze the EMG signals or NRD index (displayed in or not in graphical form) to come to a conclusion independently of the automatic prediction of the onset of the acute COPD exacerbation. In any event, in the case where onset of the acute COPD exacerbation is determined to be imminent, an alert/notification can be transmitted (whether or not prompted by the patient care provider) that the acute COPD exacerbation of the patient 12 is imminent, and that the patient 12 should be admitted to a hospital or otherwise examined by a physician.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

1. A medical monitoring system for a patient, comprising:
an implantable sensor device configured for detecting a biomarker over at least one respiratory cycle of the patient, wherein the detected biomarker is indicative of a neural respiratory drive (NRD) of the patient; and
processing circuitry configured for generating an NRD index value based on the detected biomarker.

2. The medical monitoring system of claim 1, wherein the sensor is sized to be implanted adjacent an intercostal muscle of the patient.

3. The medical monitoring system of claim 1, wherein the biomarker is an electromyogram (EMG) signal.

4. The medical monitoring system of claim 3, wherein the processing circuitry is further configured for processing the EMG signal.

5. The medical monitoring system of claim 4, wherein processing the EMG signal comprises one of sampling the EMG signal, acquiring an envelope of the EMG signal, and integrating the EMG signal.

6. The medical monitoring system of claim 4, wherein processing the EMG signal comprises filtering cardiac artifacts from the EMG signal.

7. The medical monitoring system of claim 4, wherein the processing circuitry is further configured for deriving one or more EMG data values from the EMG signal for each respiratory cycle by obtaining a peak value, and computing a root mean square (RMS) value, a quadratic mean value, or running average value of the EMG signal.

8. The medical monitoring system of claim 7, wherein the at least one respiratory cycle comprises a plurality of respiratory cycles, and wherein generating the NRD index value comprises obtaining the highest amplitude, median, or average of the EMG data values over the plurality of respiratory cycles.

9. The medical monitoring system of claim 1, wherein the sensor device is configured for detecting the biomarker during an inspiration of the at least one respiratory cycle of the patient.

10. The medical monitoring system of claim 1, wherein the at least one respiratory cycle is at least one resting respiratory cycle.

11. The medical monitoring system of claim 1, wherein the processing circuitry is further configured for determining a heart rate variability and/or respiration rate variability of the patient from the detected biomarker, and determining a validity of the detected biomarker based on the determined heart rate variability and/or respiration rate variability.

12. The medical monitoring system of claim 11, further comprising memory, wherein the processing circuitry configured for storing the NRD index value in the memory if the EMG signal is determined to be valid.

13. The medical monitoring system of claim 1, wherein the processing circuitry is configured for normalizing the NRD index value.

14. The medical monitoring system of claim 13, wherein the processing circuitry is further configured for obtaining a patient-specific reference value, wherein normalizing the NRD index value comprising computing a function of the NRD index value and the patient-specific reference value.

15. The medical monitoring system of claim 1, further comprising a display configured for graphically displaying the NRD index value to the patient.

16. The medical monitoring system of claim 1, wherein the patient suffers from a respiratory disease, the processing circuitry further configured for determining an extent of the respiratory disease based on the generated NRD index.

17. The medical monitoring system of claim 16, wherein the respiratory disease is Chronic Obstructive Pulmonary Disease (COPD).

18. The medical monitoring system of claim 16, wherein the processing circuitry is further configured for predicting an onset of an acute exacerbation of the respiratory disease in the patient based on the generated NRD index value.

19. The medical monitoring system of claim 18, wherein the processing circuitry is configured for predicting the onset of the acute exacerbation of the respiratory disease in the patient comprises comparing the NRD index value to an absolute NRD threshold value, and determining that the onset of the acute exacerbation of the respiratory disease is imminent if the NRD index value exceeds the absolute NRD threshold.

20. The medical monitoring system of claim 18, wherein the processing circuitry is configured for the onset of the acute exacerbation of the respiratory disease in the patient comprises computing an NRD index difference value between the NRD index value and a previously generated NRD index value, comparing the NRD difference value to a differential NRD threshold value, and determining that the onset of the acute exacerbation of the respiratory disease is imminent if the NRD index difference value exceeds the differential NRD threshold value.

21. The medical monitoring system of claim 18, wherein the processing circuitry is further configured for generating an alert or notification signal in response to determining that the onset of the acute exacerbation of the respiratory disease in imminent.

22. The medical monitoring system of claim 1, further comprising an external control device containing at least a portion of the processing circuitry.

23. The medical monitoring system of claim 22, further comprising a personal wireless device configured for communicating with the external control device, the personal wireless device containing another portion of the processing circuitry.

24. The medical monitoring system of claim 23, further comprising a remote computer configured for communicating with the personal wireless device via a cloud network, the remote computer containing still another portion of the processing circuitry.

25.-47. (canceled)
